(19) United States
(12) Patent Application Publication
(10) Pub. No.: US 2016/0120628 A1
(43) Pub. Date: May 5, 2016

(54) FLUTTERING EAR TAG FOR BOVINE VACCINATION

(71) Applicant: THE BOARD OF REGENTS FOR OKLAHOMA STATE UNIVERSITY, Stillwater, OK (US)

(72) Inventor: Sanjay Kapil, Stillwater, OK (US)

(21) Appl. No.: 14/896,774

(22) PCT Filed: Jun. 12, 2014
(86) PCT No.: PCT/US2014/042141

§ 371 Date: Dec. 8, 2015

Related U.S. Application Data
(60) Provisional application No. 61/833,965, filed on Jun. 12, 2013.

(51) Int. Cl. A61D 1/02; A61M 37/00

(52) U.S. Cl. CPC A61D 1/025; A61M 37/00; A61M 37/0015; A61M 2037/0023; A61M 2037/0061; A61M 2037/003; A61M 37/0084; A61M 2250/00

(57) ABSTRACT

According to an aspect of the instant invention there is provided a method of medicating or vaccinating cattle via an ear tag, ear ring or other hardware affixed to or buried within the ear of the animal.

### CROSS REFERENCE TO RELATED APPLICATION

This application claims the benefit of U.S. Provisional Application No. 61/833,965 filed Jun. 12, 2013, herein incorporated by reference in its entirety for all purposes.

### TECHNICAL FIELD

This invention relates to the general subject matter of vaccinating cattle and, more particularly, to vaccinations of cattle for bovine viral diarrhea.

### BACKGROUND OF THE INVENTION

Bovine viral diarrhea (“BVD”, hereinafter) is the number one economically significant disease in cattle worldwide. BVD is normally an infection of cattle, but it has the ability to cause infections in other animals such as pigs, sheep, goats, alpacas, deer, reindeer, and bison. BVD infections are still quite common in the U.S.A.

An infection by this virus (“BVDV”) may be difficult to recognize and only a small percentage of the animals infected will show clinical signs of infection. This virus is well known for reducing milk production and increasing the risk of death loss. It can stress adult cattle and produce abortions and birth defects in calves that are born to mothers that are infected by the virus. Its economic impact worldwide is substantial.

In spite of availability of parenteral and intra nasal commercial vaccines for BVD, the infections are still quite common in USA and many other parts of the world. Although conventional parenteral and intra nasal BVDV vaccines and methods of vaccination are available, such may not be completely effective or safe. One of the major reasons for residual infections with BVD virus is the cattle are outbred. Thus, the cattle do not respond uniformly immunologically to any vaccination. The range of immune responses can vary from no response at all to a very high level of response when such cattle are treated with the same batch of vaccines. Thus, a user friendly follow-up method is needed that allows immunological monitoring by SN (serum neutralization) tests and more importantly, safer readministration of the vaccine till the target of the immune response (protective antibody titer or higher titer) is achieved.

Further, if the data on immune response is not available, the animals will still need to be boosted for sufficient protective antibody titer. In younger cattle, due to interference from maternal antibody and many other factors, a re-administration of the vaccine is needed. As such, it would be useful if the process of boosting could happen intermittently and repeatedly.

As such, what is needed is a method and apparatus for vaccinating cattle against BVD and other diseases and/or conditions that does not suffer from the disadvantages of the prior art. Accordingly, it should now be recognized, as was recognized by the present inventor, that there exists, and has existed for some time, a very real need for a method of administering a medication that would improve on the approaches currently available for cattle.

Before proceeding to a description of the present invention, however, it should be noted and remembered that the description of the invention which follows, together with the accompanying drawings, should not be construed as limiting the invention to the examples (or embodiments) shown and described. This is so because those skilled in the art to which the invention pertains will be able to devise other forms of this invention within the ambit of the appended claims.

### SUMMARY OF THE INVENTION

According to an aspect of the instant invention, there is provided a method of vaccinating cattle for BVD or other diseases/conditions via an ear tag, earring or other hardware affixed to or buried within a bovine ear. The bovine ear is a special mobile appendage that is made principally of auricular cartilage and covered by haired skin on the outside. This aspect of the appendage makes it particularly suitable for use as described herein. Further, the outside of the pinna is anatomically dissimilar to the inside of the pinna.

According to an embodiment, there is provided a vaccination ear tag for use with cattle or other animals. In one variation, the inventive tag has as some number (e.g., one to four reservoirs) compressible reservoirs each of which could contain a different (or the same) vaccine. Clearly, more or fewer reservoirs could be provided. Each of the reservoirs is designed to be in fluid communication with one or more delivery orifices, which orifices will be embedded in the ear of a cow or other animal when the device is mounted on its subject animal. In some instances each orifice will be equipped with microneedles that are inserted into the skin of the animal during installation of the ear tag and, in other instances, micro-needles or micro-applicators might be utilized as is discussed below. To maintain the patency of the channels, a proteolytic enzyme can be added to the composition.

In some embodiments, an inventive ear tag will contain one or more reservoirs of tick acaricide/antibiotics, vaccine for BVDV, bacterium vaccines, and/or, other stable vaccine components such as Rotavirus. The long and slow acting compounds of these formulations will generally be preferred. In some embodiments, the reservoirs will be made to be compressible so that a dose of the material contained therein can be injected into the subject animal by compressing the surface of the reservoir to deliver the target titer response that is needed.

Embodiments of the invention may prove to be safer and better than conventional parenteral and nasal BVDV vaccines.

The foregoing has outlined in broad terms the more important features of the invention disclosed herein so that the detailed description that follows may be more clearly understood, and so that the contribution of the instant inventors to the art may be better appreciated. The instant invention is not to be limited in its application to the details of the construction and to the arrangements of the components set forth in the following description or illustrated in the drawings. Rather, the invention is capable of other embodiments and of being practiced and carried out in various other ways not specifically enumerated herein. Finally, it should be understood that the phraseology and terminology employed herein are for the purpose of description and should not be regarded as limiting, unless the specification specifically so limits the invention.

### BRIEF DESCRIPTION OF THE DRAWINGS

Other objects and advantages of the invention will become apparent upon reading the following detailed description and upon reference to the drawings in which:

FIG. 1A contains an embodiment of an ear tag for cattle that would be suitable for use in vaccination against BVD and other sorts of pathogenic organisms.

FIG. 1B contains a cross sectional view of a portion of the embodiment of FIG. 1A.

FIG. 2 contains an illustration of an identifying ear tag together with some of the inner structure of the bovine ear.

FIG. 3 illustrates another embodiment of the invention as it might appear when affixed to an animal ear as taught here.

FIG. 4 contains a magnified cross-sectional view of still another embodiment of the invention.

FIG. 5 contains a detailed view of a component of an embodiment of the invention.

FIG. 6 contains a magnified view of the underside (skin side) of an embodiment of the instant invention that uses micro-needles.

FIG. 7 contains a flutter ear tag embodiment consistent with the teachings herein.

### DETAILED DESCRIPTION OF THE PREFERRED EMBODIMENTS

While this invention is susceptible of embodiment in many different forms, there is shown in the drawings, and will herein be described hereinafter in detail, some specific embodiments of the instant invention. It should be understood, however, that the present disclosure is to be considered an exemplification of the principles of the invention and is not intended to limit the invention to the specific embodiments or algorithms so described.

BVDV virus has been observed by the instant inventor in heretofore unrecognized cellular and tissue locations in a BVDV infected calf. More particularly, it has not been previously recognized that such can found within the basal layers of the tongue and chondrocytes of the cartilage of the ear. This could possibly be due to the presence of a unique BVDV isolate. The prior art does not disclose infection of cells of ear cartilage by BVDV. Some other viruses such as Rous Sarcoma virus and FeLV (feline leukemia) are known to infect chondrocytes. However, infection of chondrocytes by the BVDV has not been reported heretofore.

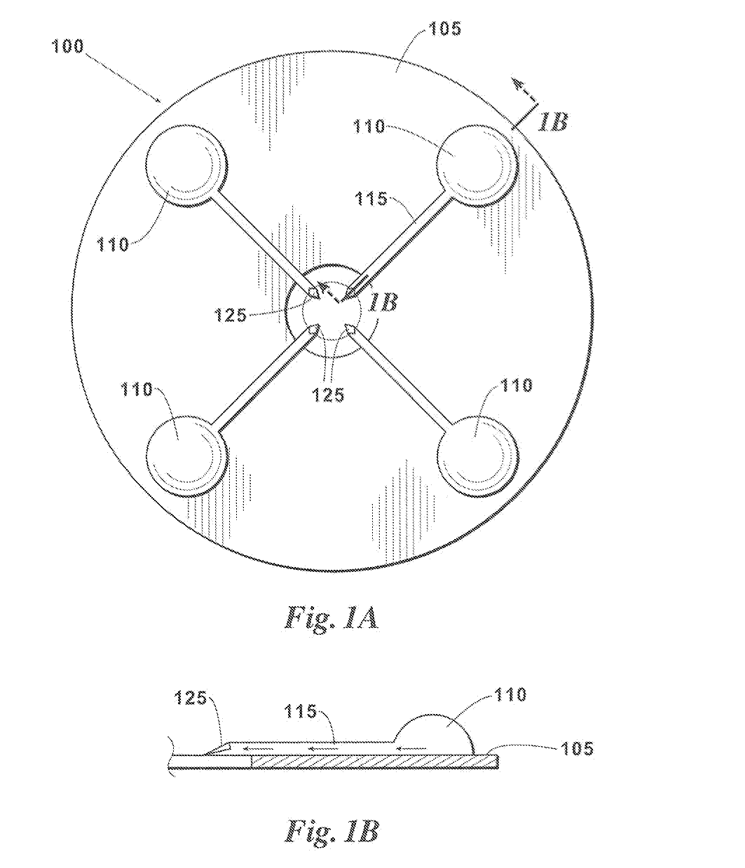

Turning next to a discussion of the example embodiments contained in the figures, FIG. 1A contains a schematic illustration of a design of the working components of an embodiment of a vaccination ear tag 100 for use with cattle or other animals. The ear tag substrate 105 might be metal or plastic or any other material that is suitable for use with animal ear tags. The tag 100 might be permanently or temporarily attached to the subject ear. In some variations, it might additionally have an RFID identification chip integral thereto or a bar code (or other visual identification scheme) printed thereon. Note that the term “tag” should be broadly construed herein and could include, for example, an ear ring in some embodiments. More generally, the term “tag” should be construed to apply to any device that is designed to be attached to and worn by an animal on its ear.

In this variation of FIG. 1A, the inventive tag 100 has four reservoirs 110 which are mounted to or supported by (permanently or temporarily) the substrate 105, each of which could contain a different (or the same) vaccine or other medication. For purposes of the instant disclosure, the term “medication” will be generally used to refer to the contents of a vaccination ear tag reservoir 110, whether or not the contents are in fact a vaccine. Clearly, more or fewer reservoirs could be provided.

FIG. 1B contains a cross sectional view of one embodiment of a reservoir 110/vaccine compartment of FIG. 1A. As can be seen, the reservoir 110 is designed to be in fluid communication (e.g., via channel 115) with one or more delivery orifices 125, which orifices will be embedded in the ear of a cow or other animal at the time when the device 100 is attached to the animal's ear. By way of example, these orifices 125 might be needles that are inserted into the skin of the animal during installation of the ear tag. In some embodiments the needle(s) used might be about 30 gauge, 25 gauge, 22 gauge, 20 gauge, and/or 18 gauge depending on the body weight or breed of cattle or, more generally, chosen depending on the type of animal to which the tag 100 is attached. In other instances and as a further example, micro-needles might be utilized as is discussed below.

In the embodiment of FIG. 1A, an ear tag 110 contains one or more reservoirs 110 that are configured to receive an amount of medication including, without limitation, tick acaricide/antibiotics, vaccine for BVDV, bacterium vaccines, and/or, other stable vaccine components such as Rotavirus. In some embodiments, the reservoirs 110 will be made to be compressible (e.g., the domed portion of the reservoir 110 in FIG. 1B might be made of a thin plastic or rubber) so that a dose of the material contained therein can be injected into the subject animal by compressing its upper surface to deliver the target titer response that is needed. Note that, for purposes of the instant disclosure, “compressible” should be understood to mean “at least somewhat compressible” in the sense that if pressure is applied to the reservoir its volume will be at least somewhat reduced, thereby forcing at least portion of its contents onto and/or into the animal's ear.

In some embodiments, the ear tag 100 might be attached to the animal's ear by any mechanism that is conventionally used to attach animal ear tags including, by way of example only, a self-piercing/locking mechanism that is integral to the tag 100 itself (e.g., if the tag is implemented in the form of a clip that is folded over the edge of the ear), a separate or integrated pin or post that passes through the ear engages and locks to a matching hole in the tag 100, etc.

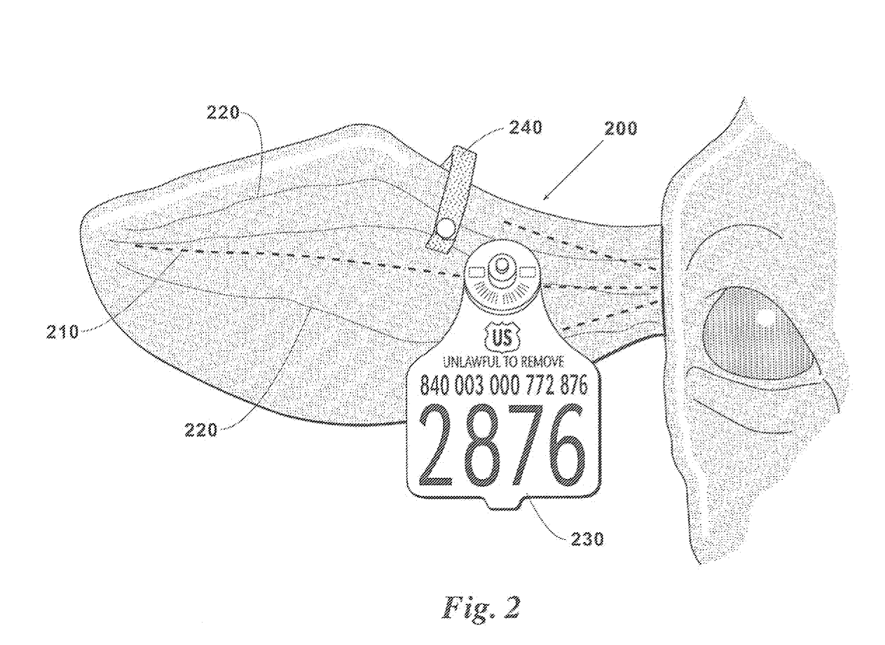

Turning next to FIG. 2, this figure illustrates in general way how an embodiment of the instant invention might be positioned and use. In this example, cow ear 200 has been drawn to indicate the presence of cartilage 210 and blood vessels 220 as they might be found in a typical cow ear. Additionally, a prior art tag 230 and a prior art USDA metal tag 240 have been used to illustrate generally where embodiments might be applied. That is, in this arrangement, an embodiment might be positioned on or proximate to a cartilaginous region of the ear 200 in a location comparable to that of the placement of a conventional ear tag 230. Additionally, in some embodiments, it would be preferably to have an embodiment placed in close proximity to one or more blood vessels 220, as is indicated in FIG. 2. Note that, although the tags in FIG. 2 are examples of prior art tags, such tags could readily be replaced by embodiments of the invention and this figure illustrates in a general way how such might be used in practice.

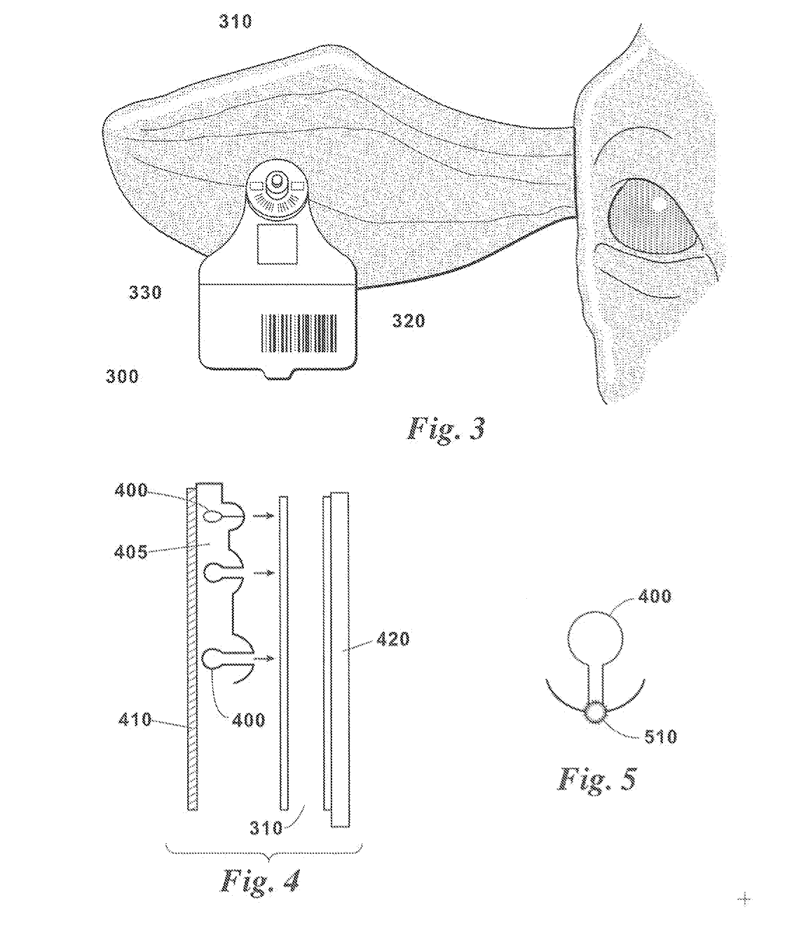

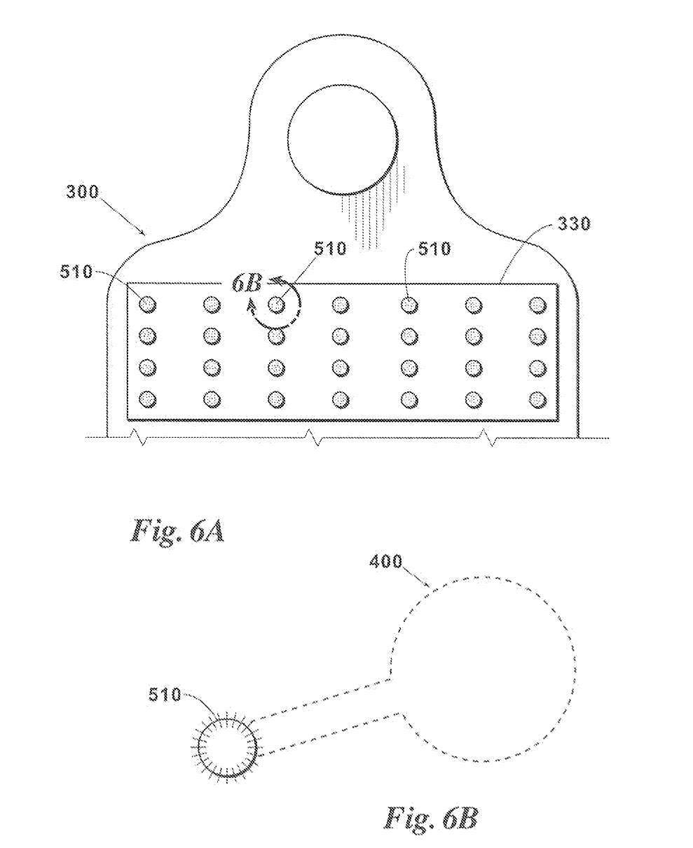

FIG. 3 is a schematic illustration of a bovine ear 310 to which has been affixed an embodiment 300. Those of ordinary skill in the art will understand that many other types of animal ears could be treated similarly. In this figure, the ear tag 300 is equipped with a bar code 320 that makes it possible to rapidly identify the particular animal and associated dosage and type of vaccination in the field. In this embodiment, and as is further indicated in FIG. 4, the application area 330 might be comprised of a plurality of vaccine depots 400 supported by a substrate 405, as is illustrated in FIG. 4 and further illustrated in FIGS. 5 and 6.

In some embodiments, each depot 400 will be designed to provide a slow release of the contents of the depot 400 into the animal to which the tag 300 has been attached. In some embodiments, the reservoir for the depot 400 might be or contain a sponge, a gel, a liquid, etc. Additionally, in some embodiments, each depot 400 will be terminated on the skinward side of the animal by micro-needles or micro-applicators 510. In some variations, this will provide an abrasive surface against which the animal's ear 310 will be placed. In such an embodiment, capillary action might be used to draw the contents of the depots 400 out and onto/into the animal's ear. Thus, the depots need not be functionally compressible, although such could be useful in certain circumstances.

The micro-needles 510 around the lip of the depot 400 in this embodiment will be designed to pierce and/or abrade the outermost layer of the skin. As has been discussed previously, intradermal delivery of the vaccine is generally as good as, if not better than, intramuscular delivery which is the typical way of vaccinating cattle and other animals. In some cases, and especially so when dealing with range cattle, the distribution of vaccine to the animal will be enhanced by the slapping and/or fluttering movement of the ears 310, with such movement tending to bring the mouth of the dispensing orifice(s)/micro needles into contact with the animal's ear and/or tending to compress/flex the associated reservoirs, thereby urging medication outward from them and into/onto the animal's skin.

FIG. 4 indicates an arrangement where the substrate 405 that supports the vaccine depots 400 has been placed on a magnetic plate or magnetic material 410 (e.g., steel), with the idea that a mating magnet or magnetic material will be placed on the opposite side of an animal's ear (e.g., magnetically attractive plate 420) thereby affixing it to the animal without piercing the ear 310. According to this example, pressure from the magnetically attractive surfaces 410/420 will tend to force the micro-applicator(s) against the ear's 310 surface, thereby causing abrasion of the surface of the ear and transmission of the contents of the depot 400 into the skin.

Finally, FIG. 6 contains a magnified view of an embodiment of a vaccination area 330. As is indicated, in this figure each of the circles 400 corresponds to the lip or mouth 510 of a depot (e.g., as such is shown in shown in FIG. 5). In such an embodiment, the associated depots 400 will preferably be at least somewhat compressible. However, that is not an absolute requirement and the contents of the depot 400 might be expelled toward the animal by capillary action.

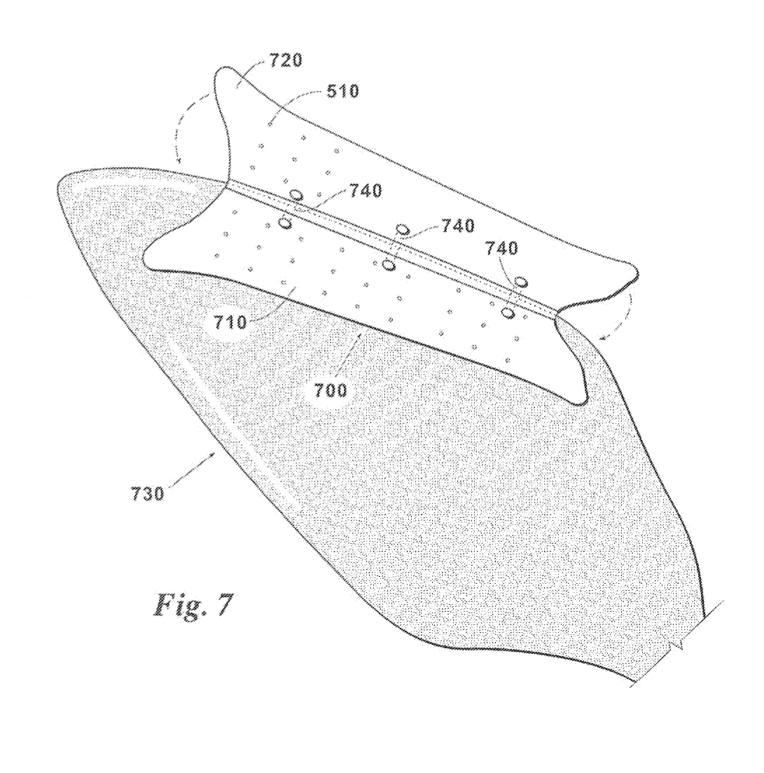

According to another embodiment and as is generally illustrated in FIG. 7, there is provided a variation that utilizes a fluttering ear tag 700. More particularly and as is set out in the example of FIG. 7, this embodiment comprises a bifurcated ear tag 700 with an upper 720 and a lower 710 wing which are folded about and loosely attached to an animal ear 730 by one or more pins 740 that pass through the ear cartilage. In the embodiment of FIG. 7, the wings are situated such that the active faces (i.e., the side containing the orifices 510) of the wings 710/720 contact the ear on opposite sides. Of course, in some embodiments there will only be a single wing or more than two wings (e.g., if each of the wings 710/720 of FIG. 7 were to be subdivided into two or more separate wings). Continuing with the present example, additionally the side on which each of the wings 710/720 contacts the animal ear 730 will be provided with some number of micro-needle orifices 510 that are designed to abrade or otherwise penetrate the outer surface of the skin as described previously each time they come into contact with the ear surface. In this example, the ear tag 700 will be made out of a lightweight plastic or other malleable material, although it could certainly be made of a rigid material such as steel, aluminum, etc.

By way of explanation, the flutter embodiment of FIG. 7 potentially has an advantage over activation by simple fanning of the ear with a securely attached tag because flutter will tend to happen more frequently and have a higher impact. However, the device of FIG. 7 will not generally be painful to the animal because micro-needles on the wings will typically be very thin and will not penetrate deeply in the ear skin. Both sides of pinna are different but the efficiency of uptake of vaccine antigens would be expected to be about as efficient from both sides of the pinna.

By way of an example, BVDV attacks both sebaceous glands that are predominant on the inner concave surface of calf pinna and also haired skin exposed to the outside. The susceptibility of vaccine antigens to light will help determine the preference of application of antigens to the outer leaf or inner leaf of the flutter ear tag 700.

The flutter ear tag 700 as taught herein can be relatively small or larger but a key feature is the exploitation of rapid and short movement distances of the tag 700 with a strike to the skin acting to terminate the movement and as a mechanism for dispensing medication to the animal. This strike movement will preferably be enhanced by loosely attaching the wings of this embodiment 700 to the ear in such a way that the contents of the depots will be deposited at slightly different points on its surface each time the wings contact the ear, thereby increasing the effective deposition area of the tag 700. This effect might further be enhanced by making the ear tags 700 out of a pliable plastic or rubber material which will tend to deform or flex when subjected to stress.

According to another embodiment, one or more springs might be positioned along the midline of the flutter apparatus 700 in such as way as to urge the wings to close toward each other after they have moved apart and away from the ear, thereby increasing the magnitude of the resulting strike against the ear and the kinetic action of the fluttering ear tag 700.

The BVDV isolate is somewhat unique in that it is very invasive and attacks some unique cell types in the ear. The cell range of the BVDV into chondrocytes makes a depot of the BVDV antigen in pinna tissue of the cattle. The inter cell movement of BVDV antigen will promote transfer and spread of BVDV antigen in the pinna tissue.

Embodiments of the disclosure taught herein will provide for an ear tag approach to vaccination. This route is user friendly because it can be combined with other cattle health related manipulations such as controlled release of vaccine antigen based on serum responses detected by BVDV SN (i.e., BVDV serum neutralization). It could allow for better vaccination coverage because the dosage of a stable BVDV vaccine in the ear tag can be manipulated and controlled based on the immune response. One BVDV isolate identified (Ser. No. 12/120,786) is unique and is a noncytopathic isolate. A BVDV antigen quantification was performed on a calf ear and the BVDV antigen was uniform in all locations of the ear and high BVDV antigen titer. Thus, embodiments of the invention are not limited by the specific position on the ear cartilage and skin of the inventive tag. IDEXX BVDV ELISA was used for these measurements of BVDV antigen. Embodiments of the invention would be compatible with DNA vaccination. For example, Bovine Lymphotropic herpes virus would be a good candidate. BLHV is known to be difficult to propagate. Thus, immunogens (gB protein and other surface proteins of BLHV) of this gammaherpes virus could be delivered as DNA vaccination.

Note that in some embodiments one reservoir might serve multiple delivery orifices. For example, in the embodiment of FIGS. 3 and 5 a single reservoir might be in fluid communication with multiple ones or all of the delivery orifices 510. Thus, it is not required that there be a one-to-one correspondence between the number of reservoirs and delivery orifices.

By way of summary, the following provides some indication of why an ear tag vaccination approach according to various embodiments of the invention might be preferred over other approaches. For example, such an approach allows a controlled release of vaccine antigen compared to parenteral vaccination and after the initial delivery there is no control. It will also provide for timed release in out bread cattle. Additionally, cattle that are out bread and respond based on the immune response genes that may not be well studied.

A method to encourage flapping of an ear mobile attachment according to the invention would be to combine an inventive embodiment with a light weight photovoltaic module. These photovoltaic modules produce their own electricity, with the current generated being used to irritate the ear, thereby encouraging the animal to flap. The lightweight nature of embodiments of the invention and the photovoltaic module(s) will not tend to discourage or impede the flapping. These small panels are low voltage and hands free to operate. Another approach that might potentially enhance the effectiveness of an embodiment would be to provide a spring or other mechanism such that, when the wind or ear movement takes the device away from the ear skin it is subsequently snapped back into place, thereby auto inoculating the animal.

Another advantage of the teachings herein is that animals on range do not have to be handled after application of the tag and this strategy potentially reduces the stress that might be associated with a vaccination. This strategy could be favorably used with wildlife, cattle and other animals at range in instances where avoiding the need to recapture the animal (e.g., for revaccination) would be beneficial. An embodiment is hands-free so boosters or repeat inoculation of vaccination happens automatically by mobility of the ear mobile attachment.

A vaccination strategy such as that taught herein might be particularly useful for extremely poor antigens or non-adjuvant vaccines. Examples of poor antigens include bacterial antigens. Most vaccines also have short duration of immunity due to lack of take of the vaccine. Duration of immunity for cattle bacterial vaccines can be very short such as 4 months for Pasteurella sp.; less than 6 months for Leptospira sp., and about 2-6 months for Clostridial antigens especially in very young cattle.

Various embodiments of the invention provide a novel way to control the release time of a drug. As an example, this might be especially useful in newborn animals. Due to colostral immunity, it is difficult to predict that when a suitable receptive window of immune response is available in a newborn animal because it can vary substantially in animals based on variables that are beyond the control of person vaccinating the animals, such as veterinarians. Additionally, generally speaking animals are brought to clinics at the convenience of the owner who may not fully appreciate or remember the need for timely vaccination, absent which there can be instances of vaccination failures. Embodiments of the invention taught herein provide a vaccination technology that potentially eliminates these sorts of variable antibody responses because it continuous can circumvent the current limitations.

Various embodiments allow easy manipulation and boosting or removal of the device as needed. The vaccine antigen or other contents of embodiments of the inventive device is deposited externally, thereby avoiding the need for implantation, oral administration, etc. Embodiments of the invention avoid problems that can be present with other/current cattle vaccine tools which may fail to properly develop immune responses to combat infections.

Various embodiments of the invention provide a needleless (needle free) approach that scarifies the ear cartilage and skin, e.g., through the use of micro needles around the deposit orifice. Skin scarification in humans has been done on arms for small pox for ages. This suggests that skin scarification is almost harmless and is not so painful as needle delivered vaccines. Cattle ears are mobile appendages unlike the human ear. Research suggests that giving a vaccine though a scratch on the skin (scarification) may trigger a stronger immune response than that obtained via an injected vaccine. Some studies suggest that scarification could require as little as 100 times less vaccine to generate an immune response as compared with an injected vaccine. All of this argues for an approach such as that taught herein.

One reason that the instant invention utilizes cartilage in some embodiments is because the vascular supply to ear cartilage is slower than elsewhere, so an antigen will tend to remain longer within cartilage and this lessens the dependence on adjuvants. There will tend to be a reduced adjuvant amount or adjuvant not needed for long-term delivery, thereby reducing side effects to vaccine associated allergies.

The ear is not an edible part so the approach taught herein may reduce issues relating to withdrawal of exposure to drugs and vaccines in food (muscle). As compared with existing methods of administering BVDV vaccines that are intramuscular, embodiments of the invention avoid engagement of professional antigen presentation cells that may be enormous in skin only and less in muscle. The immunity induction tends to affect the overall development of protective immune response development of vaccination. Additionally, cartilage is firm allowing manipulation for distribution circuit development by laser etching which can allow controlled flow of biological material. Etching can be closed and modified over life of the cattle for increased or decreased bioavailability.

An ear tag according to the instant invention could possibly also be used as a component of a total cattle management plan. For example, in one embodiment the instant invention could be used in the context of nutrition, health and vaccination, and as an insecticide delivery tool/device.

It should be noted and remembered that the instant invention might be made in a variety of different sizes, weights, etc. Further, the number, placement, and size of the teeth on the central wheel as disclosed herein might readily be modified according to the type of animal, size of animal, type of substance being delivered, etc. Those of ordinary skill in the art will understand how this might be done.

It should be further noted that ear tag embodiments of the invention are easily replaceable and the vaccine use can be monitored/adjusted for specific breed of cattle. In some embodiments, ear tags will be reloadable and removable allowing total vaccine dose delivery flexibility unlike any other vaccine system developed for cattle heretofore. An embodiment that mixes a vaccine with tattoo ink could help evaluate the distribution of the vaccine and site of antigen deposition. This could provide for site reaction and precise location of the vaccination.

If the cattle are on range or feedlot, another modification of ear tag could be applied. In this case the tag could be made to be more pliable. In seasons when there is substantial wind, the pliability modification could prove to be a useful mechanism to increase or decrease the speed with which the medication is delivered to the animal. Manipulation of the flutter activity of vaccination tag is also possible. A ferro-magnetic or magnetized vaccination tag is also possible in some embodiments. Further, remote control of ear tag movement is also possible.

Among the many modifications and extensions of the teachings provided herein includes adaptation of the ear isolates to bovine cartilage cell line. Bovine ears tend to be cooler than the core body temperature of an animal. As such, a few degree lower temperature will allow using cold-adapted BVDV viruses to be used for ear-tag vaccination according to the instant disclosure. For adapting BVD isolates for ear tag vaccination, the viruses could be propagated first in Madin-Darby bovine kidney celline or other suitable cell line such as bovine turbinate. The adaptation of BVDV could be slowly achieved by multiple passages at 36.5; 36; 35.5; 35; 34.5; 34; 33.5 and 32 F. One approach would be to achieve a BVDV natural variant that adapts to a cooler temperature without lowering the growth and BVDV titers.

Another modification of the BVDV variant would be to select BVDV variants by plaque purification. For this variation the plaque sizes could be compared with passage I (parent BVDV isolate) of the BVDV isolate that infects cartilage and skin. The BVDV isolate to be used could also be checked for infection in bovine chondrocyte cell culture. By adapting to the bovine chondrocyte the fitness for infecting the bovine cartilage and ear skin will also be validated in vitro.

Further with respect to an embodiment mentioned previously, one modification of the approaches taught herein would be to use tattoo ink with a BVDV or other vaccine or medication. Tattoo ink can potentially serve several purposes that could be beneficial for embodiments of the ear-tag drug administration system taught herein. For example, in some embodiments the ink could prevent inactivation of the BVDV virus by reducing the risk of inactivation due to exposure to the environment, e.g., exposure to sun (including UV), wind, rain, etc. In some variations, tattoo ink could be used to provide a visible marker of the site or point of BVDV or other vaccine inoculation that could be readily perceived by the naked eye. For example, the more intense the apparent color the more BVDV virus that will have been inoculated in the ear. Various color tattoo inks could be mixed together provided they do not affect the viability of the MLV BVDV or other vaccine or medication. Both surfaces of an ear could potentially be tattooed/inoculated. The effects of light/sun light might be reduced by combining an immunogen with a dark (or other appropriately chosen) tattoo ink. Wind might tend to dry the immunogen dispensed by an embodiment but it will still be antigenic. Finally, rain should not affect the immunogen deposits because such is deposited in some embodiments on the inner leaf of the ear tag.

Hepatitis C virus, a virus related to BVDV, has been transmitted in prisons by tattoo ink and tattoo procedure. Thus, BVDV, and potentially other medications, might be expected to be stable in tattoo ink. However, titration of BVDV after mixing with tattoo ink could be performed to ensure that it does not affect the BVDV viability. In some embodiments it will be possible to combine tattoo ink with the contents of a depot and/or fill some depots with ink alone (in any of a number of different colors) and fill other depots only with medication. Multiple/different tattoo ink colors might be used in some embodiments and this could be useful where multiple/different medications are included in the same ear tag. Such could also be useful where an animal is serially treated with different medications, e.g., “blue” for the first ear tag medication, “red” for a subsequent ear tag medication or subsequent application of the same medication, etc. Both soluble and even aggregated vaccine antigens are potentially applicable and suitable to confirmation by this vaccination method.

The embodiments herein and the various features and advantageous details thereof are explained more fully with reference to the non-limiting embodiments that are illustrated in the accompanying drawings and detailed in the following description. Descriptions of well-known components and processes and manufacturing techniques are omitted so as to not unnecessarily obscure the embodiments herein. The examples used herein are intended merely to facilitate an understanding of ways in which the invention herein may be practiced and to further enable those of skill in the art to practice the embodiments herein. Accordingly, the examples should not be construed as limiting the scope of the claimed invention.

Note that, for purposes of the instant disclosure, when the term “inoculated” is used that term should be broadly construed to include conventional inoculation as well as treatment, management, care, healing, etc., which involves the administration of a substance according to a claimed variation or as taught herein.

It is to be understood that the terms “including”, “comprising”, “consisting” and grammatical variants thereof do not preclude the addition of one or more components, features, steps, or integers or groups thereof and that the terms are to be construed as specifying components, features, steps or integers.

If the specification or claims refer to “an additional” element, that does not preclude there being more than one of the additional element.

It is to be understood that where the claims or specification refer to “a” or “an” element, such reference is not be construed that there is only one of that element.

It is to be understood that where the specification states that a component, feature, structure, or characteristic “may”, “might”, “can” or “could” be included, that particular component, feature, structure, or characteristic is not required to be included.

Where applicable, although state diagrams, flow diagrams or both may be used to describe embodiments, the invention is not limited to those diagrams or to the corresponding descriptions. For example, flow need not move through each illustrated box or state, or in exactly the same order as illustrated and described.

Methods of the present invention may be implemented by performing or completing manually, automatically, or a combination thereof, selected steps or tasks.

The term “method” may refer to manners, means, techniques and procedures for accomplishing a given task including, but not limited to, those manners, means, techniques and procedures either known to, or readily developed from known manners, means, techniques and procedures by practitioners of the art to which the invention belongs.

For purposes of the instant disclosure, the term “at least” followed by a number is used herein to denote the start of a range beginning with that number (which may be a ranger having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. Terms of approximation (e.g., “about”, “substantially”, “approximately”, etc.) should be interpreted according to their ordinary and customary meanings as used in the associated art unless indicated otherwise. Absent a specific definition and absent ordinary and customary usage in the associated art, such terms should be interpreted to be ±10% of the base value.

When, in this document, a range is given as “(a first number) to (a second number)” or “(a first number)-(a second number)”, this means a range whose lower limit is the first number and whose upper limit is the second number. For example, 25 to 100 should be interpreted to mean a range whose lower limit is 25 and whose upper limit is 100. Additionally, it should be noted that where a range is given, every possible subrange or interval within that range is also specifically intended unless the context indicates to the contrary. For example, if the specification indicates a range of 25 to 100 such range is also intended to include subranges such as 26-100, 27-100, etc., 25-99, 25-98, etc., as well as any other possible combination of lower and upper values within the stated range, e.g., 33-47, 60-97, 41-45, 28-96, etc. Note that integer range values have been used in this paragraph for purposes of illustration only and decimal and fractional values (e.g., 46.7-91.3) should also be understood to be intended as possible subrange endpoints unless specifically excluded.

It should be noted that where reference is made herein to a method comprising two or more defined steps, the defined steps can be carried out in any order or simultaneously (except where context excludes that possibility), and the method can also include one or more other steps which are carried out before any of the defined steps, between two of the defined steps, or after all of the defined steps (except where context excludes that possibility).

Further, it should be noted that terms of approximation (e.g., “about”, “substantially”, “approximately”, etc.) are to be interpreted according to their ordinary and customary meanings as used in the associated art unless indicated otherwise herein. Absent a specific definition within this disclosure, and absent ordinary and customary usage in the associated art, such terms should be interpreted to be plus or minus 10% of the base value.

Thus, the present invention is well adapted to carry out the objects and attain the ends and advantages mentioned above as well as those inherent therein. While presently preferred embodiments have been described for purposes of this disclosure, numerous changes and modifications will be apparent to those skilled in the art. Such changes and modifications are encompassed within the spirit of this invention as defined by the appended claims.

While the inventive device has been described and illustrated herein by reference to certain embodiments in relation to the drawings attached hereto, various changes and further modifications, apart from those shown or suggested herein, may be made therein by those skilled in the art, without departing from the spirit of the inventive concept, the scope of which is to be determined by the following claims.

What is claimed is:

1. An ear tag for vaccinating an animal with a vaccine, comprising:
(a) a substrate suitable for attachment to an ear of the animal;
(b) a compressible reservoir supported by said substrate for receiving the vaccine therein; and,
(c) a delivery orifice in fluid communication with said compressible reservoir for piercing or abrading a surface of a skin of the ear of the animal and for delivery of said vaccine from said reservoir to the ear of the animal.

2. The ear tag according to claim 1, wherein said delivery orifice comprises a hollow needle, said hollow needle having a gauge that is between about 18 gauge and about 30 gauge.

3. The ear tag according to claim 1, wherein said delivery orifice comprises one or more micro-needles.

4. The ear tag according to claim 1, wherein said delivery orifice is positionable to be placed into continuous contact with the ear of the animal.

5. The ear tag according to claim 1 wherein said substrate is suitable for permanent attachment to the ear of the animal.

6. The ear tag according to claim 1, wherein said reservoir is comprised of a material selected from the group consisting of a rubber material and a plastic material.

7. The ear tag according to claim 1, wherein said vaccine is a vaccine for BVD.

8. The ear tag according to claim 7, wherein said vaccine is a vaccine for the BVD isolate 12120786.

9. The ear tag according to claim 1, wherein the animal is selected from the group consisting of a cow, a pig, a sheep, a goat, an alpaca, a deer, a reindeer, and a bison.

10. The ear tag according to claim 1, wherein the vaccine further comprises a tattoo ink.

11. An ear tag for intradermal delivery of a medication to an animal's ear, comprising:
(a) a substrate suitable for attachment to the ear of the animal;
(b) a plurality of reservoirs supported by said substrate for receiving the medication;
(c) two or more delivery orifices, each of said two or more delivery orifices being in fluid communication one of said plurality of reservoirs, and each of said two or more delivery orifices being positionable to be placed into contact with a skin of the ear of the animal and piercing or abrading a surface of a skin of the ear of the animal for delivery of said medication from said reservoir to the ear of the animal when so positioned.

12. The ear tag according to claim 11, wherein each of said two or more delivery orifices comprises a hollow needle.

13. The ear tag according to claim 11, wherein each of said two or more delivery orifices comprises one or more micro-needles.

14. The ear tag according to claim 11, wherein each of said delivery orifices is positionable to be placed into continuous contact with the ear of the animal.

15. The ear tag according to claim 11 wherein at least one of said plurality of reservoirs contains a tattoo ink.

16. The ear tag according to claim 11 wherein at least one of said plurality of reservoirs contains a tattoo ink and the medication.

17. The ear tag according to claim 11, wherein each of said plurality of reservoirs is compressible and is comprised of a material selected from the group consisting of a rubber material and a plastic material.

18. The ear tag according to claim 11, wherein said medication is a vaccine for BVD.

19. The ear tag according to claim 11, wherein the animal is selected from the group consisting of a cow, a pig, a sheep, a goat, an alpaca, a deer, a reindeer, and a bison.

20. The ear tag according to claim 11, wherein the animal is selected from the group consisting of a cow, a pig, a sheep, a goat, an alpaca, a deer, a reindeer, and a bison.

21. The ear tag according to claim 11, wherein said ear tag is bifurcated into at least one upper wing and at least one lower wing, and wherein said bifurcated ear tag is configured to be situated on the animal ear with one or more of said at least one upper wings being on one side of the ear and one of said at least one lower wings being on an opposite side of the ear.

22. The ear tag according to claim 21, further comprising at least one spring situated between said at least one upper wing and said at least one lower wing, wherein said spring urges said at least one upper wing and said at least one lower wing toward each other when situated on the animal ear.

23. The ear tag according to claim 11, wherein at least two of said delivery orifices is in fluid communication with a same one of said plurality of reservoirs.

24. A method of administering a medication to an animal having an ear, comprising the steps of:
(a) affixing an ear tag to the ear of the animal, wherein said ear tag comprises a substrate that supports a reservoir containing the medication, said reservoir being in fluid communication with at least one delivery orifice that is supported by said substrate; and,
(b) embedding said at least one delivery orifice into the skin of the ear of the animal.

25. A method of administering a medication to an animal wherein said medication is a vaccine for BVD.

26. A method of administering a medication to an animal having an ear, comprising the steps of:
(a) treating an abrading ear tag with a medication, said abrading ear tag being configured to abrade a surface of the ear when positioned thereon; and,
(b) affixing said abrading ear tag to the ear of the animal.

27. A method of administering a medication to an animal wherein said medication further includes an amount of tattoo ink, comprising the further step of:
(c) evaluating an extent of a distribution of the tattoo ink in the ear to determine a distribution of the medication.
